(19) United States
(12) Patent Application Publication
(10) Pub. No.: US 2007/0140997 A1
(43) Pub. Date: Jun. 21, 2007

(54) Photoprotective compositions containing a triazine derivative, an amide-based oil and a eutectic mixture of n-butylphthalimide/isopropylphthalimide

(75) Inventors: Didier Candau, Bievres (FR); Cecile Fiandino, Paris (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/646,280

(22) Filed: Dec. 28, 2006

Related U.S. Application Data
(63) Continuation of application No. PCT/EP05/07888, filed on Jun. 13, 2005.

(30) Foreign Application Priority Data
Jul. 2, 2004 (FR) 04/51424

(51) Int. Cl. A61K 8/49; A61K 8/42

(52) U.S. Cl. 424/59

(57) ABSTRACT

Stable, topically applicable cosmetic/dermatological photoprotective compositions contain: a) at least one 1,3,5-triazine compound, b) at least one oil containing in its structure at least one amide structural unit, and c) at least one eutectic mixture of n-butylphthalimide and of isopropylphthalimide, and have enhanced sun protection factors and cosmetic properties.

### CROSS-REFERENCE TO PRIORITY/PCT/PROVISIONAL APPLICATIONS

This application claims priority under 35 U.S.C. § 119 of FR 04/51424, filed Jul. 2, 2004, and of Provisional Application No. 60/589,015, filed Jul. 20, 2004, and is a continuation of PCT/EP 2005/007888 filed Jun. 13, 2005 and designating the United States, published in the English language as WO 2006/003028 A1 on Jan. 12, 2006, each hereby expressly incorporated by reference and each assigned to the assignee hereof.

### BACKGROUND OF THE INVENTION

1. Technical Field of the Invention

The present invention relates to novel photoprotective compositions comprising at least one 1,3,5-triazine derivative, at least one amide-based oil and a eutectic mixture of n-butylphthalimide/isopropylphthalimide.

The present invention also relates to the various cosmetic applications of said compositions, especially for protecting the skin and/or the lips and/or the integuments against ultraviolet radiation, in particular solar radiation.

2. Description of Background and/or Related and/or Prior Art

It is known that light radiation with wavelengths of from 280 nm to 400 nm permits tanning of the human epidermis, and that rays with wavelengths of from 280 nm to 320 nm, known as UV-B rays, cause skin burns and erythema which may be harmful to the development of a natural tan; this UV-B radiation should thus be screened out.

It is also known that UV-A rays, with wavelengths of from 320 nm to 400 nm, which cause tanning of the skin, are liable to induce an adverse change in the latter, especially in the case of sensitive skin or of skin which is continually exposed to solar radiation. UV-A rays cause, in particular, a loss of elasticity of the skin and the appearance of wrinkles, leading to premature skin aging. They promote triggering of the erythemal reaction or amplify this reaction in certain individuals and may even be the cause of phototoxic or photoallergic reactions. It is thus desirable also to screen out UV-A radiation.

Many cosmetic compositions useful for the photoprotection (UV-A and/or UV-B) of the skin have been proposed to date.

These anti-sun compositions are quite often in the form of an emulsion of oil-in-water type (that is to say, a cosmetically acceptable support consisting of a continuous aqueous dispersing phase and a discontinuous oily dispersed phase) which contains, in various concentrations, one or more conventional lipophilic and/or hydrophilic organic screening agents which are capable of selectively absorbing harmful UV radiation, these screening agents (and their amounts) being selected as a function of the desired sun protection factor, the sun protection factor (SPF) being expressed mathematically by the ratio of the UV radiation dose necessary to reach the erythema-forming threshold with the UV screening agent to the UV radiation dose necessary to reach the erythema-forming threshold without UV screening agent.

1,3,5-Triazine derivatives are particularly desired in anti-sun/sunscreen cosmetics due to the fact that they are highly active in the UV-B range, and even in the UV-A range for some of these compounds depending on the nature of the substituents involved. Furthermore, they are photostable, i.e., they show little or no chemical degradation under the action of UV radiation. They are especially described in U.S. Pat. No. 4,367,390, EP-863,145, EP-517,104, EP-570,838, EP-796,851, EP-775,698, EP-878,469 and EP-933,376, and the following are known in particular:

2,4,6-tris[p-(2′-ethylhexyl-1′-oxycarbonyl)anilino]-1,3,5-triazine or “Ethylhexyl Triazone” (INCI name), marketed under the trademark “Uvinul T 150” by BASF,

2-[(p-(tert-butylamido)anilino]-4,6-bis-[(p-(2′-ethylhexyl-1′-oxycarbonyl)anilino]-1,3,5-triazine or “Diethylhexyl Butamido Triazone” (INCI name), marketed under the trademark “Uvasorb HEB” by Sigma 3V,

2,4-bis{[4-2-ethylhexyloxy)]-2-hydroxylphenyl}-6-(4-methoxyphenyl)-1,3,5-triazine or “Bis-Ethylhexyloxyphenol Methoxyphenyl Triazine” (INCI name), marketed under the trademark “Tinosorb S” by Ciba Specialty Chemicals.

It has been proposed in the prior art to use 1,3,5-triazine derivatives in oils such as esters and more particularly C₁₂-C₁₅ alkyl benzoates (“Finsolv TN” from Finetex), or triglycerides and especially C₈-C₁₂ fatty acid triglycerides (“Miglyol 812” from Hüls), or alternatively oxyethylenated or oxypropylenated fatty monoalcohols or polyols (“Cetiol HE” from Henkel or “Witconol AM” from Witco). The use of these oils presents two drawbacks:

(i) either the appearance over time of crystallization in the formulations, which is detrimental to the cosmetic qualities, the stability and the efficacy of anti-sun products;

(ii) or the limitation of the concentration of screening agents in the formulations, which does not make it possible to obtain products that are sufficiently effective.

The technical problem underlying the present invention is thus that of improving the photoprotective efficacy, the cosmetic properties and the stability of compositions containing such 1,3,5-triazine derivatives.

With this aim, it has already been proposed in EP-0,748,623 to add to 2,4,6-tris[p-(2′-ethylhexyl-1′-oxycarbonyl)anilino]-1,3,5-triazine or “Ethylhexyl Triazone” N,N-disubstituted amide-based compounds without emulsifying properties, for instance N,N-diethyl-3-methylbenzamide or ethyl N-butyl-N-acetylaminopropionate.

It has also been proposed in EP-1-269,980 to add to UV-screening agents of triazine type N-acyl esters of amino acids, for instance isopropyl N-lauroylsarcosinate.

The results obtained with these amide-based oils are still not entirely satisfactory.

It has also been proposed in EP-1430,881 to add a eutectic mixture of n-butylphthalimide/isopropylphthalimide to UV-screening agents of triazine type.

The results obtained with this eutectic mixture are still not entirely satisfactory.

### SUMMARY OF THE INVENTION

It has now surprisingly and unexpectedly been discovered that the technical problem mentioned above can be solved by means of a combination of an oil containing in its structure at least one amide structural unit and a eutectic mixture of n-butylphthalimide/isopropylphthalimide in compositions containing at least one triazine derivative.

This combination permits formulating stable anti-sun compositions containing 1,3,5-triazine derivatives, which have a sun protection factor higher than those of the prior art compositions containing 1,3,5-triazine derivatives. These compositions also have improved cosmetic qualities. They especially allow good moisturization of the skin, i.e., no dryness of the skin and no greasy feel are observed.

The present invention thus features photoprotective cosmetic or dermatological compositions containing:

a) at least one 1,3,5-triazine compound;

b) at least one oil containing in its structure at least one amide structural unit; and

c) at least one eutectic mixture of n-butylphthalimide and of isopropylphthalimide.

This invention also features the use of such compositions for the formulation of cosmetic or dermatological compositions suited in particular for protecting keratin materials against the damaging effects of solar radiation.

The present invention also features the formulation of a combination of (i) at least one oil containing in its structure at least one amide unit and (ii) a eutectic mixture of n-butylphthalimide and of isopropylphthalimide into photoprotective cosmetic or dermatological compositions containing at least one 1,3,5-triazine derivative, in order to improve the sun protection factor and/or the cosmetic qualities and/or the stability of such compositions.

Other aspects of the invention will become apparent from the description and the examples that follow.

### DETAILED DESCRIPTION OF BEST MODE AND SPECIFIC/PREFERRED EMBODIMENTS OF THE INVENTION

The expression “photoprotective composition” means any composition containing at least one organic compound and/or at least one mineral compound capable of screening out UV-A and/or UV-B radiation via a phenomenon of absorption, reflection or scattering.

The expression “oil containing in its structure at least one amide structural unit” will be understood throughout the text of the description to mean any compound comprising in its chemical structure at least one amide group (or function) of the type: 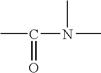
and simultaneously having the following characteristics:

a) liquid at 25° C.,

b) insoluble or immiscible in water at 25° C.,

c) no emulsifying properties.

Preferably, the combination of eutectic mixture of n-butylphthalimide and of isopropylphthalimide/amide-based oil is present in the composition in a sufficient amount to make it possible by itself to dissolve (without it being necessary to use another solvent) the total amount of triazine screening agent present in the composition.

The oil(s) having in their structure at least one amide unit in accordance with the invention is (are) preferably selected from the compounds of formula (1) below: 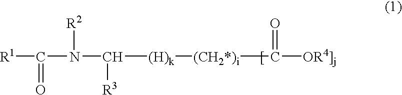
in which:

the radical R¹ is an optionally functionalized, aliphatic, cycloaliphatic or cyclic, saturated or unsaturated monovalent hydrocarbon-based radical having from 1 to 30 carbon atoms and preferably from 1 to 22 carbon atoms, inclusive;

the radicals R², R³ and R⁴, which may be identical or different, are each optionally functionalized, aliphatic, cycloaliphatic or cyclic, saturated or unsaturated monovalent hydrocarbon-based radicals having from 1 to 30 carbon atoms and preferably from 1 to 22 carbon atoms, inclusive;

k is 0 or 1;

i is an integer from 0 to 2;

j is 0 or 1;

with the proviso that:

when j=1, then k is 0 and when j=0, then i=0 and k=1.

Examples of saturated aliphatic hydrocarbon-based radicals that are especially representative include linear or branched, substituted or unsubstituted C₁-C₃₀ and preferably C₁-C₂₂ alkyl radicals, and in particular methyl, ethyl, n-propyl, isopropyl, n-butyl, isobutyl, tert-butyl, pentyl, n-amyl, isoamyl, neopentyl, n-hexyl, n-heptyl, n-octyl, 2-ethylhexyl, tert-octyl, decyl, lauryl and octadecyl radicals.

Examples of saturated cyclic hydrocarbon-based radicals that are especially representative include cyclopentyl and cyclohexyl radicals, which are optionally substituted, in particular with alkyl radicals.

Examples of unsaturated aliphatic hydrocarbon-based radicals that are especially representative include linear or branched, substituted or unsubstituted, C₂-C₃₀ and preferably C₂-C₂₂ alkenyl or alkynyl radicals, and in particular vinyl, allyl, oleyl and linoleyl radicals.

Examples of unsaturated cyclic hydrocarbon-based radicals that are especially representative include aryl radicals such as phenyl and naphthyl, which are optionally substituted, in particular with alkyls, for instance a tolyl radical, and examples of unsaturated cycloaliphatic radicals that are more particularly representative include benzyl and phenylethyl radicals.

The term “functionalized radicals” more particularly means radicals comprising in their chemical structure, either in the main chain or on a secondary chain unit, one or more functional groups especially such as esters, ethers, alcohols, amines, amides and ketones, but preferably esters.

The preferred amide-based oils of formula (1) are selected from those in which:

R¹ is a linear or branched C₁-C₂₂ alkyl radical; a linear or branched C₂-C₂₂ alkenyl radical; an aryl radical;

R² is a hydrogen atom or a linear or branched C₁-C₆ alkyl radical;

R³ is a hydrogen atom or a linear or branched C₁-C₆ alkyl radical;

R⁴ is a linear or branched C₁-C₁₀ alkyl radical or a linear or branched C₂-C₁₀ alkenyl radical or a sterol residue.

In formula (1) presented above, the group R¹(CO)— is an acyl group of an acid preferably selected from the group consisting of acetic acid, toluic acid, capric acid, lauric acid, myristic acid, palmitic acid, stearic acid, behenic acid, linoleic acid, linolenic acid, oleic acid, isostearic acid, 2-ethylhexanoic acid, coconut oil fatty acids and palm kernel oil fatty acids. These acids may also contain a hydroxyl group.

In formula (1), when j is 1, the moiety —N(R²)CH(R³)(CH₂)_{q}(CO)— of the amino acid ester is preferably selected from those corresponding to the following amino acids:

glycine, alanine, valine, leucine, isoleucine, serine, threonine, proline, hydroxyproline, β-alanine, N-butyl-β-alanine, aminobutyric acid, aminocaproic acid, sarcosine or N-methyl-β-alanine.

In formula (1), when j is 1, the moiety of the amino acid esters corresponding to the group OR⁴ may be obtained from alcohols selected from the group consisting of methanol, ethanol, propanol, isopropanol, butanol, tert-butanol, isobutanol, 3-methyl-1-butanol, 2-methyl-1-butanol, pentanol, hexanol, cyclohexanol, octanol, 2-ethylhexanol, decanol, lauryl alcohol, myristyl alcohol, cetyl alcohol, cetostearyl alcohol, stearyl alcohol, oleyl alcohol, behenyl alcohol, jojoba alcohol, 2-hexadecyl alcohol, 2-octyldodecanol and isostearyl alcohol.

The oils containing in their structure at least one amide function of formula (1) in accordance with the invention are known per se. Certain of these are especially described with their methods of preparation in EP-1-044,676 and EP-0,928,608 of the company Ajinomoto Co. Others are known in cosmetics, for instance insect repellents such as ethyl N-acetyl-N-butylaminopropionate or N,N-diethyltoluamide.

Among the compounds of formula (1) that are particularly preferred, exemplary are:
ethyl N-acetyl-N-butylaminopropionate, having the following formula: 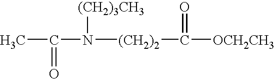
such as the product marketed under the trademark Repellent R3535 by Merck;
isopropyl N-lauroylsarcosinate of formula: 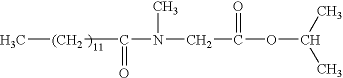
such as the product marketed under the trademark Eldew SL-205 by Ajimoto;
N,N-diethyltoluamide of formula: 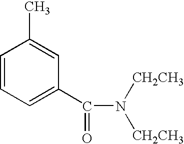
such as the product marketed under the trademark Deet by Showa Denko.

The oil(s) containing in their structure at least one amide function as defined above is (are) present in the compositions according to the invention in concentrations preferably ranging from 0.1% to 40% by weight and more preferably from 1% to 20% by weight relative to the total weight of the composition.

The eutectic mixture in accordance with the invention preferably contains:

(a) 60% to 75% by weight of n-butylphthalimide of structure (2) below:

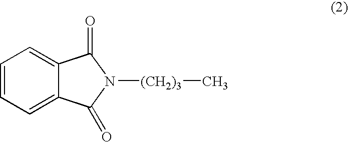

(b) 25% to 40% by weight of isopropylphthalimide in accordance with the invention, corresponding to structure (3) below:

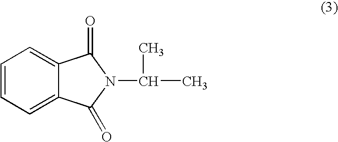

The n-butylphthalimide/isopropylphthalimide eutectic mixtures in accordance with the invention are known and are described, as is the synthesis thereof, in U.S. Pat. No. 6,306,373 (hereby expressly incorporated by reference).

Among the eutectic mixtures according to the invention, the following mixtures are exemplary:

n-butylphthalimide/isopropylphthalimide (60/40% by weight)

n-butylphthalimide/isopropylphthalimide (62/38% by weight)

n-butylphthalimide/isopropylphthalimide (65/35% by weight)

n-butylphthalimide/isopropylphthalimide (70/30% by weight)

n-butylphthalimide/isopropylphthalimide (75/25% by weight).

The n-butylphthalimide/isopropylphthalimide eutectic mixture marketed under the trademark Pelemol BIP by Phoenix Chemicals is exemplary.

The eutectic mixture in accordance with the invention will preferably be formulated at concentrations ranging from 0.1% to 40% by weight and more preferably from 1% to 30% by weight relative to the total weight of the composition.

The 1,3,5-triazine compound corresponds to formula (I) below: 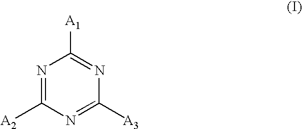
in which the radicals A₁, A₂ and A₃, which may be identical or different, are each selected from the groups of formulae (II) to (IX) below: 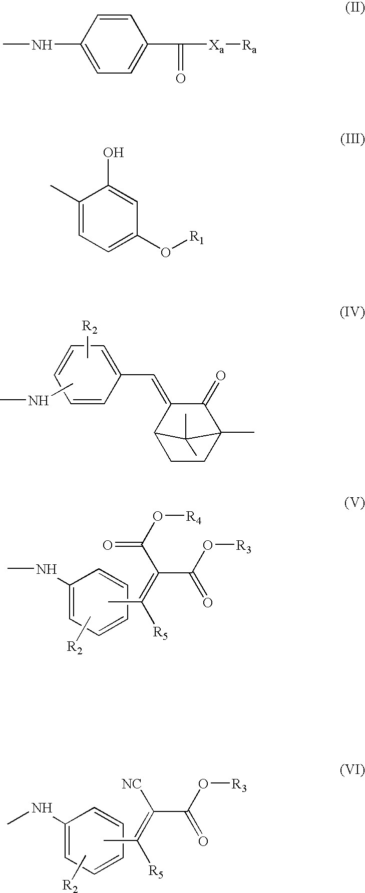 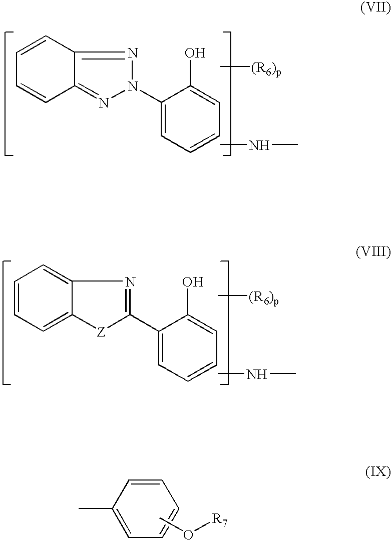
in which:

Xₐ (each of the groups Xₐ may be identical or different) is oxygen or —NH—;

Rₐ (each of the groups Rₐ may be identical or different) is selected from among hydrogen; an alkali metal; an ammonium radical optionally substituted with one or more linear or branched C₁-C₁₈ alkyl or linear or branched C₁-C₁₈ hydroxyalkyl radicals; a linear or branched C₁-C₁₈ and preferably C₆-C₁₂ alkyl radical; a C₅-C₁₂ cycloalkyl radical optionally substituted with one or more C₁-C₄ alkyl radicals; a polyoxyethylenated radical containing from 1 to 6 ethylene oxide units, the terminal OH group of which is methylated; a radical of formula (X), (XI) or (XII) below: 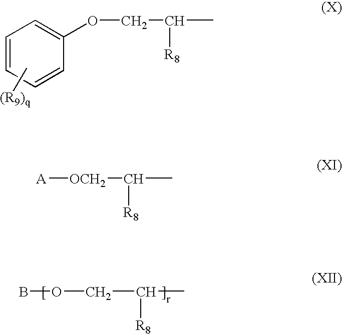
in which:

R₈ is hydrogen or a methyl radical;

R₉ is a C₁-C₉ alkyl radical;

q is an integer equal to 0; 1; 2; 3;

r is an integer equal to 1; 2; 3; 4; 5; 6; 7; 8; 9; 10;

A is a C₄-C₈ alkyl radical or a C₅-C₈ cycloalkyl radical;

B is selected from among a linear or branched C₁-C₈ alkyl radical; a C₅-C₈ cycloalkyl radical; an aryl radical optionally substituted with one or more C₁-C₄ alkyl radicals;

R₁ is a C₃-C₁₈ alkyl radical; a C₂-C₁₈ alkenyl radical; a residue of formula —CH₂—CH(OH)—CH₂—OT₁ in which T₁ is a hydrogen atom or a C₁-C₈ alkyl radical; a residue of formula (XIII) below: 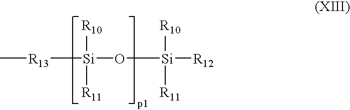
in which:

R₁₃ is a covalent bond; a linear or branched C₁-C₄ alkyl radical or a radical of formula —Cₘ₁H₂ₘ₁—O— in which m₁ is an integer equal to 1; 2; 3; 4;

p₁ is an integer equal to 0; 1; 2; 3; 4; 5;

the radicals R₁₀, R₁₁ and R₁₂, which may be identical or different, are each a C₁-C₁₈ alkyl radical; a C₁-C₁₈ alkoxy radical or a radical of formula: 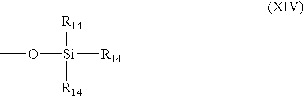
in which R₁₄ is a C₁-C₅ alkyl radical;
R₂ is a hydrogen atom, a linear or branched C₁-C₄ alkyl radical or a C₁-C₄ alkoxy radical;

R₃ and R₄, which may be identical or different, are each a linear or branched C₁-C₂₀ alkyl radical;

R₅ is a hydrogen atom or a phenyl radical optionally substituted with a halogen or with a C₁-C₄ alkyl radical or with a C₁-C₄ alkoxy radical;

R₆ is a linear or branched C₁-C₈ alkyl radical or a C₁-C₃ alkoxy radical, with the proviso that, in the latter case, two adjacent radicals R₆ on the same aromatic nucleus can together form an alkylidenedioxy radical in which the alkylidene group contains 1 or 2 carbon atoms, OH, NHCOCH₃ or NH₂;

R₇ is a hydrogen atom, a C₁-C₁₀ alkyl radical, a radical of formula: —(CH₂CHR₅—O)ₙ₁R₈ in which n₁ is a number from 1 to 16, or a radical of structure —CH₂—CH—(OH)—CH₂OT₁ wherein R₈ and T₁ having the same meaning as indicated above;

Z is oxygen, sulfur, —NH— or —NR₃— wherein R₃ is a linear or branched C₁-C₂₀ alkyl radical;

p is 0, 1, 2 or 3; with the proviso that

A₁ can also be a halogen, a radical —N(R₃)₂, wherein the two radicals R₃ may together form a ring of 4 or 5 carbon atoms, or a group —OR₃, wherein R₃ has the same definition as above.

A first family of 1,3,5-triazine derivatives that is more particularly preferred, and that is described especially in EP-A-0,517,104, is that of the 1,3,5-triazines corresponding to formula (I) in which A₁, A₂ and A₃ are of formula (II) and have all of the following characteristics:

one of the groups Xₐ-Rₐ is a radical-NH-Rₐ wherein Rₐ is selected from among a C₅-C₁₂ cycloalkyl radical optionally substituted with one or more C₁-C₄ alkyl radicals; a radical of formula (X), (XI) or (XII) above in which:

B is a C₁-C₄ alkyl radical;

R₉ is a methyl radical;

the other two groups Xₐ-Rₐ represent a radical —O—Rₐ wherein the radicals Rₐ, which may be identical or different, are selected from among hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which:

B is a C₁-C₄ alkyl radical;

R₉ is a methyl radical.

A second family of 1,3,5-triazine derivatives that is more particularly preferred, and that is described especially in EP-A-0,570,838, is that of the 1,3,5-triazines corresponding to formula (I) in which A₁, A₂ and A₃ are of formula (II) and have all of the following characteristics:

one or two groups Xₐ-Rₐ represent a radical —NH-Rₐ, wherein the radicals Rₐ are selected from among a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which B is a C₁-C₄ alkyl radical and R₉ is a methyl radical;

the other or the other two group(s) Xₐ-Rₐ being a radical —O-Rₐ wherein the radicals Rₐ, which may be identical or different, are selected from among hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which B is a C₁-C₄ alkyl radical and R₉ is a methyl radical.

A 1,3,5-triazine of this second family that is particularly preferred is 2-[(p-(tert-butylamido)anilino]-4,6-bis[(p-(2′-ethylhexyl-1′-oxycarbonyl)anilino]-1,3,5-triazine or “Diethylhexyl Butamido Triazone” marketed under the trademark “Uvasorb HEB” by Sigma 3V and corresponding to the following formula: 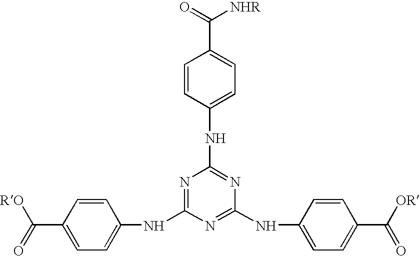
in which R′ is a 2-ethylhexyl radical and R is a tert-butyl radical.

A third preferred family of compounds according to the present invention, and which is described especially in U.S. Pat. No. 4,724,137, is that of the 1,3,5-triazines corresponding to formula (I) in which A₁, A₂ and A₃ are of formula (II) and have all of the following characteristics:

Xₐ are identical and represent oxygen;

Rₐ, which may be identical or different, represent a C₆-C₁₂ alkyl radical or a polyoxyethylenated radical comprising from 1 to 6 ethylene oxide units, the terminal OH group of which is methylated.

A 1,3,5-triazine of this third family that is particularly preferred is 2,4,6-tris[p(2′-ethylhexyl-1′-oxycarbonyl)anilino]-1,3,5-triazine or “Ethylhexyl Triazone” marketed especially under the trademark “Uvinul T 150” by BASF and corresponds to the following formula: 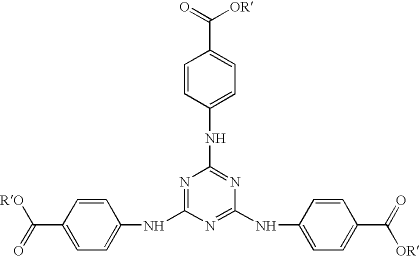
in which R′ is a 2-ethylhexyl radical.

A fourth preferred family of compounds according to the present invention, and which is described especially in EP-A-0,775,698, is that of the 1,3,5-triazines corresponding to formula (I) in which A₁ and A₂ are of formula (III) and A₃ is of formula (IX) and have all of the following characteristics: R₁, which may be identical or different, are each a C₃-C₁₈ alkyl radical; a C₂-C₁₈ alkenyl radical or a residue of formula —CH₂—CH(OH)—CH₂—OT₁ in which T₁ is a hydrogen atom or a C₁-C₈ alkyl radical; R₇ is a hydrogen atom or a C₁-C₁₀ alkyl radical.

A 1,3,5-triazine of this fourth family that is particularly preferred is 2,4-bis{[4-2-ethylhexyloxy)]-2-hydroxylphenyl}-6-(4-methoxyphenyl)-1,3,5-triazine or “Anisotriazine” marketed under the trademark “Tinosorb S” by Ciba Specialty Chemicals, and corresponds to the following formula: 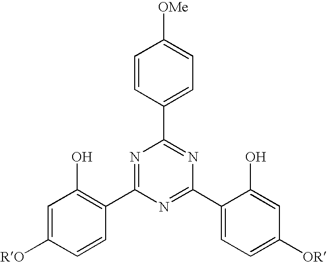
in which R′ is a 2-ethylhexyl radical.

A fifth preferred family of compounds according to the present invention, and which is described especially in EP-507,691, EP-507,692, EP-790,243 and EP-944,624, and the technical content of which is incorporated in its entirety in the present description by reference, is that of the 1,3,5-triazines corresponding to formula (I) in which A₁, A₂ and A₃ are of formulae (VII) to (XI) mentioned above.

As examples of these compounds, representative are:
2,4,6-tris(diisobutyl 4′-aminobenzalmalonate)-s-triazine,
2,4,6-tris(bis(2-ethylhexyl) 4′-aminobenzalmalonate)-s-triazine,
2,4,6-tris(bis(2-ethylhexyl) 4′-aminobenzalmalonate)-6-chloro-s-triazine,
2,4,6-tris(bis(2-ethylhexyl) 4′-aminobenzalmalonate)-6-(2-ethylhexyl 4′-aminobenzoate)-s-triazine,
2,4,6-tris(diisobutyl 4′-aminobenzalmalonate)-6-butoxy-s-triazine,
2,4,6-tris(diisobutyl 4′-aminobenzalmalonate)-6-(2-ethylhexylamino)-s-triazine,
2,4-bis(4′-aminobenzylidenecamphor)-6-(2-ethylhexylamino)-s-triazine,
2,4-bis(4′-aminobenzylidenecamphor)-6-(diisobutyl 4′-aminobenzalmalonate)-s-triazine,
2,4,6-tris(diethyl 4′-aminobenzalmalonate)-s-triazine,
2,4,6-tris(diisopropyl 4′-aminobenzalmalonate)-s-triazine,
2,4,6-tris(dimethyl 4′-aminobenzalmalonate)-s-triazine,
2,4,6-tris(ethyl α-cyano-4-aminocinnamate)-s-triazine,
2,4,6-tris[(3′-benzotriazol-2-yl-2′-hydroxy-5′-methyl)phenylamino]-s-triazine,
2,4,6-tris[(3′-benzotriazol-2-yl-2′-hydroxy-5′-tert-octyl)phenylamino]-s-triazine.

A 1,3,5-triazine of this fifth family that is particularly preferred is 2,4,6-tris(diisobutyl 4′-aminobenzalmalonate)-s-triazine, which corresponds to the following formula:

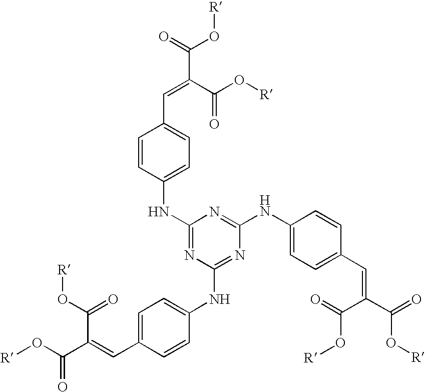

The compositions according to the present invention preferably comprise, in a physiologically acceptable medium, from 0.05% to 15% and preferably from 0.1% to 10% of 1,3,5-triazine derivatives by weight relative to the total weight of the said composition.

The compositions according to the present invention are preferably cosmetic compositions containing, other than the 1,3,5-triazine derivative as organic screening agent, at least one other additional organic screening agent and/or at least one other additional mineral screening agent, which is water-soluble, liposoluble or insoluble in the cosmetic solvents commonly used.

The compositions in accordance with the invention may also comprise other additional UV-A-active and/or UV-B-active organic or mineral photoprotective agents that are water-soluble or liposoluble or insoluble in the cosmetic solvents commonly used.

The additional organic photoprotective agents are selected especially from among anthranilates; cinnamic derivatives; salicylic derivatives; camphor derivatives; benzophenone derivatives; β,β-diphenylacrylate derivatives; benzotriazole derivatives; benzalmalonate derivatives; benzimidazole derivatives; imidazolines; bis-benzazolyl derivatives as described in EP-669,323 and U.S. Pat. No. 2,463,264; p-aminobenzoic acid (PABA) derivatives; methylenebis(hydroxyphenylbenzotriazole) derivatives as described in U.S. Pat. Nos. 5,237,071, 5,166,355, GB-2,303,549, DE-197,26,184 and EP-893,119; benzoxazole derivatives as described in EP-0,832,642, EP-1,027,883, EP-1,300,137 and DE-101,62,844; screening polymers and screening silicones such as those described especially in WO 93/04665; dimers derived from α-alkylstyrene, such as those described in DE-198,55,649; 4,4-diarylbutadienes such as those described in EP-0,967,200, DE-197,46,654, DE-197,55,649, EP-A-1,008,586, EP-1,133,980 and EP-133,981, and mixtures thereof.

As examples of additional organic screening agents, representative are those denoted hereinbelow under their INCI name:

Para-Aminobenzoic Acid Derivatives:

PABA,

Ethyl PABA,

Ethyl dihydroxypropyl PABA,

Ethylhexyl dimethyl PABA marketed in particular under the trademark

“Escalol 507” by ISP,

Glyceryl PABA,

PEG-25 PABA marketed under the trademark “Uvinul P25” by BASF.

Salicylic Derivatives:

Homosalate marketed under the trademark “Eusolex HMS” by Rona/EM Industries,

Ethylhexyl salicylate marketed under the trademark “Neo Heliopan OS” by Haarmann and Reimer,

Dipropylene glycol salicylate marketed under the trademark “Dipsal” by Scher,

TEA salicylate marketed under the trademark “Neo Heliopan TS” by Haarmann and Reimer.

Cinnamic Derivatives:

Ethylhexyl methoxycinnamate marketed in particular under the trademark “Parsol MCX” by Hoffmann LaRoche,

Isopropyl methoxycinnamate,

Isoamyl methoxycinnamate marketed under the trademark “Neo Heliopan E 1000” by Haarmann and Reimer,

Cinoxate,

DEA methoxycinnamate,

Diisopropyl methylcinnamate,

Glyceryl ethylhexanoate dimethoxycinnamate.

Dibenzoylmethane Derivatives:

Butyl methoxydibenzoylmethane marketed in particular under the trademark “Parsol 1789” by Hoffmann LaRoche,

Isopropyldibenzoylmethane.

β,β-Diphenylacrylate Derivatives:

Octocrylene marketed in particular under the trademark “Uvinul N539” by BASF,

Etocrylene marketed in particular under the trademark “Uvinul N35” by BASF.

Benzophenone Derivatives:

Benzophenone-1 marketed under the trademark “Uvinul 400” by BASF,

Benzophenone-2 marketed under the trademark “Uvinul D50” by BASF,

Benzophenone-3 or Oxybenzone marketed under the trademark “Uvinul M40” by BASF,

Benzophenone-4 marketed under the trademark “Uvinul MS40” by BASF,

Benzophenone-5,

Benzophenone-6 marketed under the trademark “Helisorb 11” by Norquay,

Benzophenone-8 marketed under the trademark “Spectra-Sorb UV-24” by American Cyanamid,

Benzophenone-9 marketed under the trademark “Uvinul DS-49” by BASF,

Benzophenone-12

n-hexyl 2-(4-diethylamino-2-hydroxybenzoyl)benzoate.

Benzylidenecamphor Derivatives:

3-Benzylidenecamphor manufactured under the trademark “Mexoryl SD” by Chimex,

4-Methylbenzylidenecamphor marketed under the trademark “Eusolex 6300” by Merck,

Benzylidenecamphorsulfonic acid manufactured under the trademark “Mexoryl SL” by Chimex,

Camphor benzalkonium methosulfate manufactured under the trademark “Mexoryl SO” by Chimex,

Terephthalylidenedicamphorsulfonic acid manufactured under the trademark “Mexoryl SX” by Chimex,

Polyacrylamidomethylbenzylidenecamphor manufactured under the trademark “Mexoryl SW” by Chimex.

Phenylbenzimidazole Derivatives:

Phenylbenzimidazolesulfonic acid marketed in particular under the trademark “Eusolex 232” by Merck,

Disodium phenyldibenzimidazoletetrasulfonate marketed under the trademark “Neo Heliopan AP” by Haarmann and Reimer.

Phenylbenzotriazole Derivatives:

Drometrizole trisiloxane marketed under the trademark “Silatrizole” by Rhodia Chimie,

Methylenebis(benzotriazolyl)tetramethylbutylphenol marketed in solid form under the trademark “MIXXIM BB/100” by Fairmount Chemical, or in micronized form as an aqueous dispersion under the trademark “Tinosorb M” by Ciba Specialty Chemicals.

Anthranilic Derivatives:

Menthyl anthranilate marketed under the trademark “Neo Heliopan MA” by Haarmann and Reimer.

Imidazoline Derivatives:

Ethylhexyldimethoxybenzylidenedioxoimidazoline propionate.

Benzalmalonate Derivatives:

Polyorganosiloxane containing benzalmalonate functions, for instance Polysilicone-15, marketed under the trademark “Parsol SLX” by Hoffmann LaRoche

4,4-Diarylbutadiene Derivatives:

1,1-Dicarboxy(2,2′-dimethylpropyl)-4,4-diphenylbutadiene,

Benzoxazole Derivatives:

2,4-bis[5-1 (dimethylpropyl)benzoxazol-2-yl(4-phenyl)imino]-6-(2-ethylhexyl)imino-1,3,5-triazine marketed under the trademark Uvasorb K2A by Sigma 3V

and mixtures thereof.

The preferred additional organic UV-screening agents are selected from among:

Homosalate,

Ethylhexyl salicylate,

Ethylhexyl methoxycinnamate,

Butylmethoxydibenzoylmethane,

Octocrylene,

Phenylbenzimidazolesulfonic acid,

Benzophenone-3,

Benzophenone-4,

Benzophenone-5,

n-Hexyl 2-(4-diethylamino-2-hydroxybenzoyl)benzoate,

4-Methylbenzylidenecamphor,

Terephthalylidenedicamphorsulfonic acid,

Disodium phenyldibenzimidazoletetrasulfonate,

Methylenebis(benzotriazolyl)tetramethylbutylphenol,

Drometrizole trisiloxane,

Polysilicone-15,

1,1-Dicarboxy(2,2′-dimethylpropyl)-4,4-diphenylbutadiene,

2,4-Bis[5-1 (dimethylpropyl)benzoxazol-2-yl(4-phenyl)imino]-6-(2-ethylhexyl)imino-1,3,5-triazine,

and mixtures thereof.

The additional mineral photoprotective agents are selected from among pigments and even more preferably nanopigments (mean size of the primary particles: generally from 5 nm to 100 nm to preferably from 10 nm to 50 nm) of treated or untreated metal oxides such as, for example, nanopigments of titanium oxide (amorphous or crystallized in rutile and/or anatase form), or of iron oxide, zinc oxide, zirconium oxide or cerium oxide.

The treated nanopigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal (titanium or aluminum) alkoxides, polyethylene, silicones, proteins (collagen or elastin), alkanolamines, silicon oxides, metal oxides, sodium hexametaphosphate, alumina or glycerol.

The treated nanopigments may more particularly be titanium oxides treated with:

silica and alumina, such as the products “Microtitanium Dioxide MT 500 SA” and “Microtitanium dioxide MT 100 SA” from the company Tayca, and the products “Tioveil Fin”, “Tioveil OP”, “Tioveil MOTG” and “Tioveil IPM” from the company Tioxide,

alumina and aluminum stearate, such as the product “Microtitanium Dioxide MT 100 T” from the company Tayca,

alumina and aluminum laurate, such as the product “Microtitanium Dioxide MT 100 S” from the company Tayca,

iron oxides and iron stearate, such as the product “Microtitanium Dioxide MT 100 F” from the company Tayca,

silica, alumina and silicone, such as the products “Microtitanium Dioxide MT 100 SAS”, “Microtitanium Dioxide MT 600 SAS” and “Microtitanium Dioxide MT 500 SAS” from the company Tayca,

sodium hexametaphosphate, such as the product “Microtitanium Dioxide MT 150 W” from the company Tayca,

octyltrimethoxysilane, such as the product “T-805” from the company Degussa,

alumina and stearic acid, such as the product “UVT-M160” from the company Kemira,

alumina and glycerol, such as the product “UVT-M212” from the company Kemira,

alumina and silicone, such as the product “UVT-M262” from the company Kemira.

Other titanium oxide nanopigments treated with a silicone are preferably TiO₂ treated with octyltrimethylsilane and for which the mean size of the elementary particles is from 25 to 40 nm, such as the product marketed under the trademark “T 805” by Degussa Silices, TiO₂ treated with a polydimethylsiloxane and for which the mean size of the elementary particles is 21 nm, such as the product marketed under the trademark “70250 Cardre UF TiO2SI3” by Cardre, anatase/rutile TiO₂ treated with a polydimethylhydrogenosiloxane and for which the mean size of the elementary particles is 25 nm, such as the product marketed under the trademark “Microtitanium Dioxide USP Grade Hydrophobic” by Color Techniques.

The uncoated titanium oxide nanopigments are marketed, for example, by Tayca under the trademarks “Microtitanium Dioxide MT 500 B” or “Microtitanium Dioxide MT 600 B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”.

The uncoated zinc oxide nanopigments are, for example:

those marketed under the trademark “Z-Cote” by Sunsmart;

those marketed under the trademark “Nanox” by Elementis;

those marketed under the trademark “Nanogard WCD 2025” by Nanophase Technologies.

The coated zinc oxide nanopigments are, for example:

those marketed under the trademark “Zinc Oxide CS-5” by Toshibi (ZnO coated with polymethylhydrogenosiloxane);

those marketed under the trademark “Nanogard Zinc Oxide FN” by Nanophase Technologies (as a 40% dispersion in Finsolv TN, C₁₂-C₁₅ alkyl benzoate);

those marketed under the trademark “Daitopersion ZN-30” and “Daitopersion ZN-50” by Daito (dispersions in cyclopolymethylsiloxane/oxyethylenated polydimethylsiloxane, containing 30% or 50% of nanozinc oxides coated with silica and polymethylhydrogenosiloxane);

those marketed under the trademark “NFD Ultrafine ZNO” by Daikin (ZnO coated with perfluoroalkyl phosphate and copolymer based on perfluoroalkylethyl as a dispersion in cyclopentasiloxane);

those marketed under the trademark “SPD-Z1” by Shin-Etsu (ZnO coated with silicone-grafted acrylic polymer, dispersed in cyclodimethylsiloxane);

those marketed under the trademark “Escalol Z100” by ISP (alumina-treated ZnO dispersed in an ethylhexyl methoxycinnamate/PVP-hexadecene copolymer/methicone mixture);

those marketed under the trademark “Fuji ZNO-SMS-10” by Fuji Pigment (ZnO coated with silica and polymethylsilsesquioxane);

those marketed under the trademark “Nanox Gel TN” by Elementis (ZnO dispersed at a concentration of 55% in C₁₂-C₁₅ alkyl benzoate with hydroxystearic acid polycondensate).

The uncoated cerium oxide nanopigments are marketed under the trademark “Colloidal Cerium Oxide” by Rhone-Poulenc.

The uncoated iron oxide nanopigments are marketed, for example, by Arnaud under the trademarks “Nanogard WCD 2002 (FE 45B)” and “Nanogard Iron FE 45 BL AQ”, “Nanogard FE 45R AQ”, “Nanogard WCD 2006 (FE 45R)” or by Mitsubishi under the trademark “TY-220”.

The coated iron oxide nanopigments are marketed, for example, by Arnaud under the trademarks “Nanogard WCD 2008 (FE 45B FN)”, “Nanogard WCD 2009 (FE 45B 556)”, “Nanogard FE 45 BL 345” and “Nanogard FE 45 BL” or by BASF under the trademark “Transparent Iron Oxide”.

Also exemplary are mixtures of metal oxides, especially of titanium dioxide and of cerium dioxide, including the silica-coated equal-weight mixture of titanium dioxide and of cerium dioxide, marketed by Ikeda under the trademark “Sunveil A”, and also the alumina, silica and silicone-coated mixture of titanium dioxide and of zinc dioxide, such as the product “M 261” marketed by Kemira, or the alumina, silica and glycerol-coated mixture of titanium dioxide and of zinc dioxide, such as the product “M 211” marketed by Kemira.

The nanopigments may be introduced into the compositions according to the invention in unmodified form or in the form of pigmentary paste, i.e., as a mixture with a dispersant, as described, for example, in GB-A-2,206,339.

The additional photoprotective agents are generally present in the compositions according to the invention in proportions ranging from 0.01% to 20% by weight relative to the total weight of the composition and preferably ranging from 0.1% to 10% by weight relative to the total weight of the composition.

The compositions according to the invention may also contain agents for artificially tanning and/or browning the skin (self-tanning agents) and more particularly dihydroxyacetone (DHA). They are preferably present in amounts ranging from 0.1% to 10% by weight relative to the total weight of the composition.

The compositions in accordance with the present invention may also comprise standard cosmetic adjuvants selected especially from among fatty substances, organic solvents, ionic or nonionic, hydrophilic or lipophilic thickeners, softeners, humectants, opacifiers, stabilizers, emollients, silicones, anti-foams, fragrances, preservatives, anionic, cationic, nonionic, zwitterionic or amphoteric surfactants, active agents, fillers, polymers, propellants, acidifying or basifying agents or any other ingredient usually used in cosmetics and/or dermatology.

The fatty substances may be an oil or a wax or mixtures thereof. The term “oil” means a compound that is liquid at room temperature. The term “wax” means a compound that is solid or substantially solid at room temperature and whose melting point is generally greater than 35° C.

Oils that are exemplary include mineral oils (paraffin); plant oils (sweet almond oil, macadamia oil, grapeseed oil or jojoba oil); synthetic oils, for instance perhydrosqualene, fatty alcohols, fatty acids or fatty esters (for instance the C₁₂-C₁₅ alkyl benzoate marketed under the trademark “Finsolv TN” by Witco, octyl palmitate, isopropyl lanolate and triglycerides, including capric/caprylic acid triglycerides), oxyethylenated or oxypropylenated fatty esters and ethers; silicone oils (cyclomethicone and polydimethylsiloxanes, or PDMS) or fluoro oils, and polyalkylenes.

Waxy compounds that are exemplary include paraffin, carnauba wax, beeswax and hydrogenated castor oil.

Among the organic solvents that are exemplary are lower alcohols and polyols. These polyols may be selected from glycols and glycol ethers, for instance ethylene glycol, propylene glycol, butylene glycol, dipropylene glycol or diethylene glycol.

Hydrophilic thickeners that are exemplary include carboxyvinyl polymers such as the Carbopol products (carbomers) and the Pemulen products (acrylate/C10-C30-alkylacrylate copolymer); polyacrylamides, for instance the crosslinked copolymers marketed under the trademarks Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulfonic acid polymers and copolymers, which are optionally crosslinked and/or neutralized, for instance the poly(2-acrylamido-2-methylpropanesulfonic acid) marketed by Hoechst under the trademark “Hostacerin AMPS” (CTFA name: ammonium polyacryidimethyltauramide); cellulose-based derivatives such as hydroxyethylcellulose; polysaccharides and especially gums such as xanthan gum; and mixtures thereof.

Lipophilic thickeners that are exemplary include modified clays such as hectorite and its derivatives, for instance the products marketed under the trademark Bentone.

Among the active agents that are exemplary are:

anti-pollution agents and/or free-radical scavengers;

depigmenting agents and/or propigmenting agents;

anti-glycation agents;

NO-synthase inhibitors;

agents for stimulating the synthesis of dermal or epidermal macromolecules and/or for preventing their degradation;

agents for stimulating fibroblast proliferation;

agents for stimulating keratinocyte proliferation;

muscle relaxants;

tensioning agents;

desquamating agents;

moisturizers;

anti-inflammatory agents;

agents acting on the energy metabolism of cells;

insect repellants;

substance P or CGRP antagonists.

Needless to say, one skilled in this art will take care to select the optional additional compound(s) mentioned above and/or the amounts thereof such that the advantageous properties intrinsically associated with the compositions in accordance with the invention are not, or are not substantially, adversely affected by the envisaged addition(s).

The compositions according to the invention may be prepared according to techniques that are well known to those skilled in the art, in particular those intended for the preparation of emulsions of oil-in-water or water-in-oil type. They may be in particular in the form of a simple or complex emulsion (O/W, W/O, O/W/O or W/O/W emulsion) such as a cream or a milk, in the form of a gel or a cream-gel, or in the form of a lotion, a powder or a solid stick, and may optionally be packaged as an aerosol and may be in the form of a mousse or a spray.

The compositions according to the invention are preferably in the form of an oil-in-water or water-in-oil emulsion.

The emulsions generally contain at least one emulsifier selected from among amphoteric, anionic, cationic and nonionic emulsifiers, which are used alone or as a mixture. The emulsifiers are appropriately selected according to the emulsion to be obtained (W/O or O/W).

As emulsifying surfactants that may be used for the preparation of the W/O emulsions, examples include sorbitan, glycerol or sugar alkyl esters or ethers; silicone surfactants, for instance dimethicone copolyols, such as the mixture of cyclomethicone and of dimethicone copolyol, marketed under the trademark “DC 5225 C” by Dow Corning, and alkyldimethicone copolyols such as laurylmethicone copolyol marketed under the trademark “Dow Corning 5200 Formulation Aid” by Dow Corning; cetyldimethicone copolyol, such as the product marketed under the trademark Abil EM 90R by Goldschmidt, and the mixture of cetyidimethicone copolyol, of polyglyceryl isostearate (4 mol) and of hexyl laurate, marketed under the trademark Abil WE 09 by Goldschmidt. One or more co-emulsifiers may also be added thereto, which may be selected advantageously from the group comprising polyol alkyl esters. Polyol alkyl esters that may especially be exemplary include glycerol and/or sorbitan esters, for example polyglyceryl isostearate, such as the product marketed under the trademark Isolan GI 34 by Goldschmidt, sorbitan isostearate, such as the product marketed under the trademark Arlacel 987 by ICI, sorbitan glyceryl isostearate, such as the product marketed under the trademark Arlacel 986 by ICI, and mixtures thereof.

For the O/W emulsions, examples of emulsifiers that are exemplary include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate; fatty alkyl ethers of sugars, especially alkylpolyglucosides (APG) such as decylglucoside and laurylglucoside marketed, for example, by Henkel under the respective names Plantaren 2000 and Plantaren 1200, cetostearyl glucoside optionally as a mixture with cetostearyl alcohol, marketed, for example, under the trademark Montanov 68 by SEPPIC, under the trademark Tegocare CG90 by Goldschmidt and under the trademark Emulgade KE3302 by Henkel, and also arachidyl glucoside, for example in the form of a mixture of arachidyl alcohol, behenyl alcohol and arachidyl glucoside, marketed under the trademark Montanov 202 by SEPPIC. According to one particular embodiment of the invention, the mixture of the alkylpolyglucoside as defined above with the corresponding fatty alcohol may be in the form of a self-emulsifying composition as described, for example, in WO-A-92/06778.

When it is an emulsion, the aqueous phase of this emulsion may comprise a nonionic vesicular dispersion prepared according to known processes (Bangham, Standish and Watkins, J. Mol. Biol., 13, 238 (1965), FR-2,315,991 and FR-2,416,008).

The compositions according to the invention find their application in a large number of treatments, whether regime or regimen, especially cosmetic treatments, of the skin, the lips and the hair, including the scalp, especially for protecting and/or caring for the skin, the lips and/or the hair, and/or for making up the skin and/or the lips.

The present invention also features the use of the subject compositions as defined above for the manufacture of products for cosmetically treating the skin, the lips, the nails, the hair, the eyelashes, the eyebrows and/or the scalp, especially care products and makeup products.

The cosmetic compositions according to the invention may be administered, for example, as care products and/or anti-sun/sunscreen products for the face and/or the body, of liquid to semi-liquid consistency, such as milks, more or less rich creams, cream-gels and pastes. They may optionally be packaged as an aerosol and may be in the form of mousses or sprays.

The compositions according to the invention in the form of vaporizable fluid lotions are topically applied to the skin or the hair in the form of fine particles by means of pressurization devices. The devices in accordance with the invention are well known to those skilled in the art and include non-aerosol pumps or “atomizers”, aerosol containers comprising a propellant and also aerosol pumps using compressed air as propellant. The latter pumps are described in U.S. Pat. Nos. 4,077,441 and 4,850,517 (incorporated by reference herein).

The compositions packaged in aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

In order to further illustrate the present invention and the advantages thereof, the following specific examples of sunscreen formulations are given, it being understood that same are intended only as illustrative and in nowise limitative. In said examples to follow, all parts and percentages are given by weight, unless otherwise indicated.

| | | |
|---|---|---|
| Composition | Example 1 | Example 2 |

PHASE A

| | | |
|---|---|---|
| Polydimethylsiloxane | 0.5 | 0.5 |
| Preservatives | 1.0 | 1.0 |
| Stearic acid | 1.5 | 1.5 |
| Glyceryl monostearate/PEG stearate mixture | 1.0 | 1.0 |
| (100 EO) | | |
| Cetylstearyl glucoside/cetylstearyl alcohol | 2.0 | 2.0 |
| mixture | | |
| Cetyl alcohol | 0.5 | 0.5 |
| N-Lauroyl isopropyl sarcosinate (Eldew SL-205 - | 10 | 10 |
| Ajinomoto) | | |
| Butylphthalimide/isopropylphthalimide eutectic | 10 | 10 |
| mixture | | |
| Bis(ethylhexyloxyphenol)methoxyphenyltriazine | 5 | — |
| (Tinosorb S from Ciba Geigy) | | |
| Ethylhexyl Triazone (Uvinul T150 from BASF) | — | 5 |

PHASE B

| | | |
|---|---|---|
| Deionized water | qs 100 | qs 100 |
| Sequestering agent | 0.1 | 0.1 |
| Glycerol | 5.0 | 5.0 |
| Xanthan gum | 0.2 | 0.2 |
| Monocetyl phosphate | 1.0 | 1.0 |

PHASE C

| | | |
|---|---|---|
| Isohexadecane | 1.0 | 1.0 |
| Acrylic acid/stearyl methacrylate copolymer | 0.2 | 0.2 |
| Triethanolamine | qs | qs |

The aqueous phase (Phase B) containing all of its ingredients is heated to 80° C. in a water bath. The fatty phase (Phase A) containing all of its ingredients is heated to 80° C. in a water bath. A is emulsified in B with stirring of rotor-stator type (machine from the company Moritz). Phase C is incorporated and the mixture is allowed to cool to room temperature with moderate stirring. The triethanolamine is introduced so as to adjust the pH to the desired value at the end of manufacture.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A stable, topically applicable photoprotective cosmetic/dermatological composition, comprising:
a) at least one 1,3,5-triazine compound,
b) at least one oil containing in its structure at least one amide structural unit, and
c) at least one eutectic mixture of n-butylphthalimide and of isopropylphthalimide.

2. The photoprotective composition as defined by claim 1, in which the at least one oil containing in its structure at least one amide structural unit is selected from among the compounds of formula (1) below: 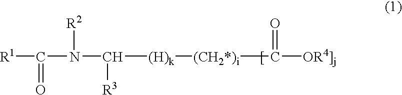
in which:
the radical R¹ is an optionally functionalized, aliphatic, cycloaliphatic or cyclic, saturated or unsaturated monovalent hydrocarbon-based radical having from 1 to 30 carbon atoms;
the radicals R², R³ and R⁴, which may be identical or different, are each an optionally functionalized, aliphatic, cycloaliphatic or cyclic, saturated or unsaturated monovalent hydrocarbon-based radical having from 1 to 30 carbon atoms;
k is 0 or 1;
i is an integer from 0 to 2;
j is 0 or 1;
with the proviso that:
when j=1, then k is 0 and when j=0, then i=0 and k=1.

3. The photoprotective composition as defined by claim 2, in which the at least one oil containing in its structure at least one amide structural unit of formula (1) is selected from among those in which:
R¹ is a linear or branched C₁-C₂₂ alkyl radical; a linear or branched C₂-C₂₂ alkenyl radical; or an aryl radical;
R² is a hydrogen atom or a linear or branched C₁-C₆ alkyl radical;
R³ is a hydrogen atom or a linear or branched C₁-C₆ alkyl radical;
R⁴ is a linear or branched C₁-C₁₀ alkyl radical or a linear or branched C₂-C₁₀ alkenyl radical or a sterol residue.

4. The photoprotective composition as defined by claim 3, in which the at least one oil containing in its structure at least one amide structural unit of formula (1) is selected from among:
ethyl N-acetyl-N-butylaminopropionate, having the following formula: 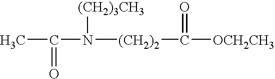
isopropyl N-lauroylsarcosinate of formula: 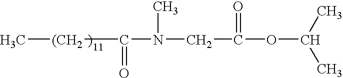
N,N-diethyltoluamide of formula:

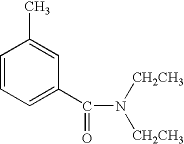

5. The photoprotective composition as defined by claim 1, in which the oil(s) containing in their structure at least one amide function is (are) present in concentrations ranging from 0.1% to 40% by weight relative to the total weight of the composition.

6. The photoprotective composition as defined by claim 1, in which the eutectic mixture comprises:
(a) 60% to 75% by weight of n-butylphthalimide of structure (2) below: 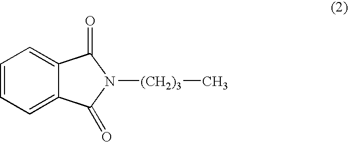
(b) 25% to 40% by weight of isopropylphthalimide of structure (3) below:

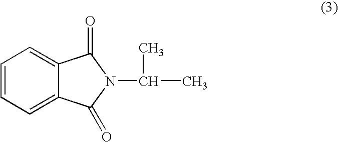

7. The photoprotective composition as defined by claim 6, in which the eutectic mixture comprises:
n-butylphthalimide/isopropylphthalimide (60/40% by weight);
n-butylphthalimide/isopropylphthalimide (62/38% by weight);
n-butylphthalimide/isopropylphthalimide (65/35% by weight);
n-butylphthalimide/isopropylphthalimide (70/30% by weight); or
n-butylphthalimide/isopropylphthalimide (75/25% by weight).

8. The photoprotective composition as defined by claim 1, in which the eutectic mixture is present in concentrations ranging from 0.1% to 50% by weight relative to the total weight of the composition.

9. The photoprotective composition as defined by claim 1, in which the at least one 1,3,5-triazine compound has the formula (I) below: 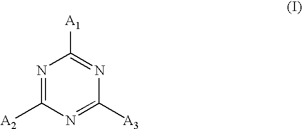
in which the radicals A₁, A₂ and A₃, which may be identical or different, are each selected from among the radicals of formulae (II) to (IX) below: 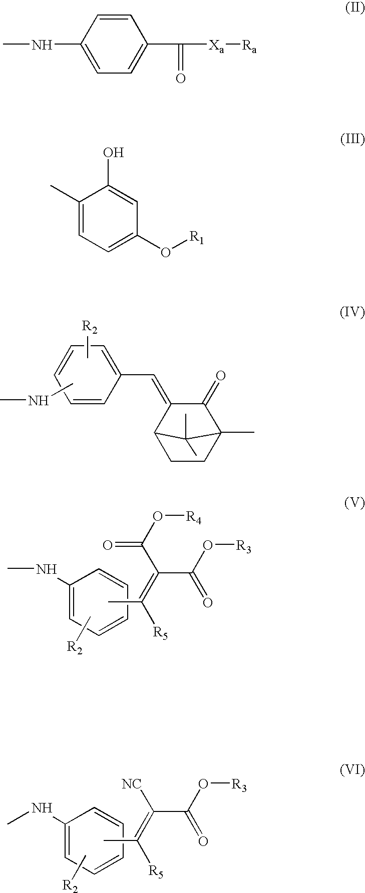 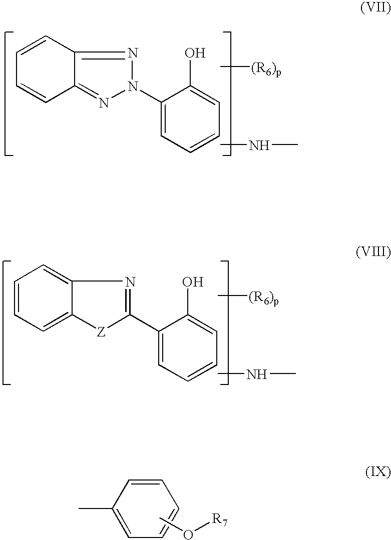
in which:
Xₐ (each of the groups Xₐ may be identical or different) is oxygen or —NH—;
Rₐ (each of the groups Rₐ may be identical or different) is selected from among hydrogen; an alkali metal; an ammonium radical optionally substituted with one or more linear or branched C₁-C₁₈ alkyl or linear or branched C₁-C₁₈ hydroxyalkyl radicals; a linear or branched C₁-C₁₈ radical; a C₅-C₁₂ cycloalkyl radical optionally substituted with one or more C₁-C₄ alkyl radicals; a polyoxyethylenated radical containing from 1 to 6 ethylene oxide units, the terminal OH group of which is methylated; a radical of formula (X), (XI) or (XII) below: 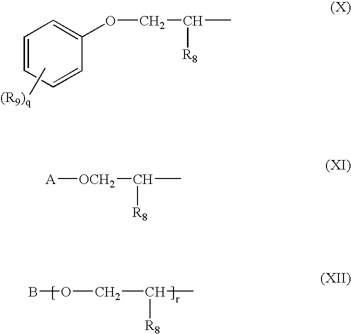
in which:
R₈ is hydrogen or a methyl radical;
R₉ is a C₁-C₉ alkyl radical;
q is an integer equal to 0; 1; 2; 3;
r is an integer equal to 1; 2; 3; 4; 5; 6; 7; 8; 9; 10;
A is a C₄-C₈ alkyl radical or a C₅-C₈ cycloalkyl radical;
B is selected from among a linear or branched C₁-C₈ alkyl radical; a C₅-C₈ cycloalkyl radical; an aryl radical optionally substituted with one or more C₁-C₄ alkyl radicals;
R₁ is a C₃-C₁₈ alkyl radical; a C₂-C₁₈ alkenyl radical; a residue of formula —CH₂—CH(OH)—CH₂—OT₁ in which T₁ is a hydrogen atom or a C₁-C₈ alkyl radical; a residue of formula (XIII) below: 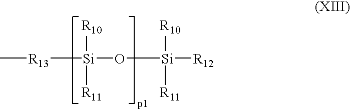
in which:
R₁₃ is a covalent bond; a linear or branched C₁-C₄ alkyl radical or a radical of formula —Cₘ₁H₂ₘ₁—O— in which m₁ is an integer equal to 1; 2; 3; 4;
p₁ is an integer equal to 0; 1; 2; 3; 4; 5;
the radicals R₁₀, R₁₁ and R₁₂, which may be identical or different, are each a C₁-C₁₈ alkyl radical; a C₁-C₁₈ alkoxy radical or a radical of formula: 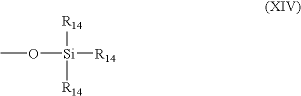
in which R₁₄ is a C₁-C₅ alkyl radical;
R₂ is a hydrogen atom, a linear or branched C₁-C₄ alkyl radical or a C₁-C₄ alkoxy radical;
R₃ and R₄, which may be identical or different, are each a linear or branched C₁-C₂₀ alkyl radical;
R₅ is a hydrogen atom or a phenyl radical optionally substituted with a halogen or with a C₁-C₄ alkyl radical or with a C₁-C₄ alkoxy radical;
R₆ is a linear or branched C₁-C₈ alkyl radical or a C₁-C₃ alkoxy radical, with the proviso that, in the latter case, two adjacent radicals R₆ on the same aromatic nucleus can together form an alkylidenedioxy group in which the alkylidene group contains 1 or 2 carbon atoms, OH, NHCOCH₃ or NH₂,
R₇ is a hydrogen atom, a C₁-C₁₀ alkyl radical, a radical of formula: —(CH₂CHR₅—O)ₙ₁R₈ in which n₁ is a number ranging from 1 to 16, or a radical of structure —CH₂—CH—(OH)—CH₂OT₁ with R₈ and T₁ having the same definitions as indicated above,
Z is oxygen, sulfur, —NH— or —NR₃— wherein R₃ is a linear or branched C₁-C₂₀ alkyl radical;
p is 0, 1, 2 or 3; and
A₁ can also be a halogen, a radical —N(R₃)₂, with the proviso that the two radicals R₃ may together form a ring member of 4 or 5 carbon atoms, or a group —OR₃, wherein R₃ has the same definition as above.

10. The photoprotective composition as defined by claim 9, in which the at least one 1,3,5-triazine compound has the formula (I) in which A₁, A₂ and A₃ are of formula (II) and have all of the following characteristics:
one of the groups Xₐ-Rₐ is a radical-NH-Rₐ wherein Rₐ is selected from among a C₅-C₁₂ cycloalkyl radical optionally substituted with one or more C₁-C₄ alkyl radicals; a radical of formula (X), (XI) or (XII) above in which:
B is a C₁-C₄ alkyl radical;
R₉ is a methyl radical;
the other two groups Xₐ-Rₐ are each a radical —O-Rₐ wherein the radicals Rₐ, which may be identical or different, are each hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which:
B is a C₁-C₄ alkyl radical;
R₉ is a methyl radical.

11. The photoprotective composition as defined by claim 9, in which the at least one 1,3,5-triazine compound has the formula (I) in which A₁, A₂ and A₃ are of formula (II) and have all of the following characteristics:
one or two groups Xₐ-Rₐ is (are) a radical —NH-Rₐ, wherein Rₐ is selected from among a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which:
B is a C₁-C₄ alkyl radical;
R₉ is a methyl radical;
the other or the other two group(s) Xₐ-Rₐ being a radical —O-Rₐ wherein the radicals Rₐ, which may be identical or different, are each hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which:
B is a C₁-C₄ alkyl radical;
R₉ is a methyl radical.

12. The photoprotective composition as defined by claim 11, in which the at least one 1,3,5-triazine compound comprises 2-[(p-(tert-butylamido)anilino]-4,6-bis[(p-(2′-ethylhexyl-1′-oxycarbonyl)anilino]-1,3,5-triazine of the following formula: 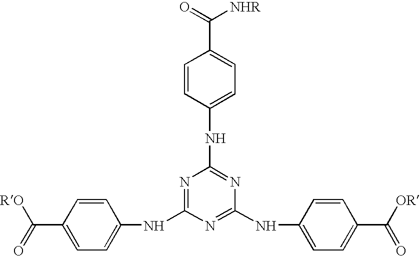
in which R′ is a 2-ethylhexyl radical and R is a tert-butyl radical.

13. The photoprotective composition as defined by claim 9, in which the at least one 1,3,5-triazine compound has the formula (I) in which A₁, A₂ and A₃ are of formula (II) and have all of the following characteristics:
the groups Xₐ are identical and are each oxygen;
the radicals Rₐ, which may be identical or different, are each a C₆-C₁₂ alkyl radical or a polyoxyethylenated radical containing from 1 to 6 ethylene oxide units, the terminal OH group of which is methylated.

14. The photoprotective composition as defined by claim 13, in which the at least one 1,3,5-triazine compound comprises 2,4,6-tris[p(2′-ethylhexyl-1′-oxycarbonyl)anilino]-1,3,5-triazine of the following formula: 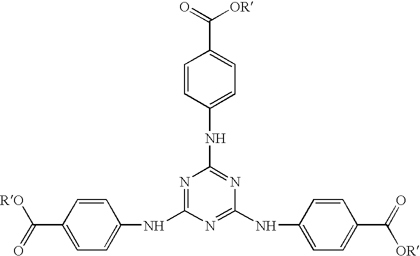
in which R′ is a 2-ethylhexyl radical.

15. The photoprotective composition as defined by claim 9, in which the at least one 1,3,5-triazine compound has the formula (I) in which A₁ and A₂ are of formula (III) and A₃ is of formula (IX) and have all of the following characteristics: the radicals R₁, which may be identical or different, are each a C₃-C₁₈ alkyl radical; a C₂-C₁₈ alkenyl radical or a residue of formula —CH₂—CH(OH)—CH₂—OT₁ in which T₁ is a hydrogen atom or a C₁-C₈ alkyl radical; and R₇ is a hydrogen atom or a C₁-C₁₀ alkyl radical.

16. The photoprotective composition as defined by claim 15, in which the at least one 1,3,5-triazine compound comprises 2,4-bis{[4-2-ethylhexyloxy)]-2-hydroxylphenyl}-6-(4-methoxyphenyl)-1,3,5-triazine of the following formula: 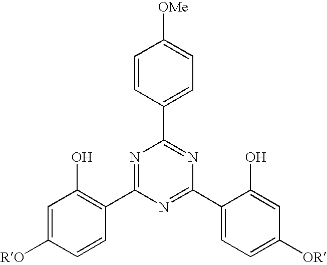
in which R′ is a 2-ethylhexyl radical.

17. The photoprotective composition as defined by claim 9, in which the at least one 1,3,5-triazine compound has the formula (I) in which A₁, A₂ and A₃ are of formulae (VII) to (XI).

18. The photoprotective composition as defined by claim 17, in which the at least one 1,3,5-triazine compound is selected from the group consisting of:
2,4,6-tris(diisobutyl 4′-aminobenzalmalonate)-s-triazine,
2,4,6-tris(bis(2-ethylhexyl) 4′-aminobenzalmalonate)-s-triazine,
2,4,6-tris(bis(2-ethylhexyl) 4′-aminobenzalmalonate)-6-chloro-s-triazine,
2,4,6-tris(bis(2-ethylhexyl) 4′-aminobenzalmalonate)-6-(2-ethylhexyl 4′-aminobenzoate)-s-triazine,
2,4,6-tris(diisobutyl 4′-aminobenzalmalonate)-6-butoxy-s-triazine,
2,4,6-tris(diisobutyl 4′-aminobenzalmalonate)-6-(2-ethylhexylamino)-s-triazine,
2,4-bis(4′-aminobenzylidenecamphor)-6-(2-ethylhexylamino)-s-triazine,
2,4-bis(4′-aminobenzylidenecamphor)-6-(diisobutyl 4′-aminobenzalmalonate)-s-triazine,
2,4,6-tris(diethyl 4′-aminobenzalmalonate)-s-triazine,
2,4,6-tris(diisopropyl 4′-aminobenzalmalonate)-s-triazine,
2,4,6-tris(dimethyl 4′-aminobenzalmalonate)-s-triazine,
2,4,6-tris(ethyl α-cyano-4-aminocinnamate)-s-triazine,
2,4,6-tris[(3′-benzotriazol-2-yl-2′-hydroxy-5′-methyl)phenylamino]-s-triazine,
2,4,6-tris[(3′-benzotriazol-2-yl-2′-hydroxy-5′-tert-octyl)phenylamino]-s-triazine, and mixtures thereof.

19. The photoprotective composition as defined by claim 1, in which the at least one 1,3,5-triazine compound is present in concentrations ranging from 0.05% to 15% by weight relative to the total weight of the said composition.

20. The photoprotective composition as defined by claim 1, further comprising at least one additional organic photoprotective agent and/or at least one additional mineral photoprotective agent, which is water-soluble, liposoluble or insoluble in the cosmetic solvents commonly employed.

21. The photoprotective composition as defined by claim 20, comprising additional organic photoprotective agents selected from among anthranilates; dibenzoylmethane derivatives; cinnamic derivatives; salicylic derivatives; camphor derivatives; benzophenone derivatives; β,β′-diphenylacrylate derivatives; benzotriazole derivatives; benzalmalonate derivatives; benzimidazole derivatives; imidazolines; bis-benzazolyl derivatives; p-aminobenzoic acid (PABA) derivatives; methylenebis(hydroxyphenylbenzotriazole) derivatives; benzoxazole derivatives; screening polymers and screening silicones; dimers derived from α-alkylstyrene; 4,4-diarylbutadienes, and mixtures thereof.

22. The photoprotective composition as defined by claim 21, comprising additional organic screening agents selected from among:
Homosalate,
Ethylhexyl salicylate,
Ethylhexyl methoxycinnamate,
Butylmethoxydibenzoylmethane,
Octocrylene,
Phenylbenzimidazolesulfonic acid,
Benzophenone-3,
Benzophenone-4,
Benzophenone-5,
n-Hexyl 2-(4-diethylamino-2-hydroxybenzoyl)benzoate,
4-Methylbenzylidenecamphor,
Terephthalylidenedicamphorsulfonic acid,
Disodium phenyldibenzimidazoletetrasulfonate,
Methylenebis(benzotriazolyl)tetramethylbutylphenol,
Drometrizole trisiloxane,
Polysilicone-15,
1,1-Dicarboxy(2,2′-dimethylpropyl)-4,4-diphenylbutadiene,
2,4-Bis[5-1 (dimethylpropyl)benzoxazol-2-yl(4-phenyl)imino]-6-(2-ethylhexyl)imino-1,3,5-triazine,
and mixtures thereof.

23. The photoprotective composition as defined by claim 20, comprising additional mineral photoprotective agents selected from among coated or uncoated metal oxide pigments or nanopigments.

24. The photoprotective composition as defined by claim 23, comprising additional mineral screening agents that are nanopigments of titanium oxide, which is amorphous or crystallized in rutile and/or anatase form, or of iron oxide, zinc oxide, zirconium oxide or cerium oxide.

25. The photoprotective composition as defined by claim 1, further comprising at least one active agent for artificially tanning and/or browning the skin.

26. The photoprotective composition as defined by claim 1, further comprising at least one adjuvant selected from among fatty substances, organic solvents, ionic or nonionic, hydrophilic or lipophilic thickeners, softeners, humectants, opacifiers, stabilizers, emollients, silicones, anti-foams, fragrances, preservatives, anionic, cationic, nonionic, zwitterionic or amphoteric surfactants, active agents, fillers, polymers, propellants and acidifying or basifying agents.

27. The photoprotective composition as defined by claim 1, wherein the combination of eutectic mixture of n-butylphthalimide and of isopropylphthalimide/amide-based oil is present therein in an amount that is sufficient for dissolving by itself all of the triazine screening agent present in the composition.

28. The photoprotective composition as defined by claim 1, formulated as a product for cosmetically treating the skin, the lips, the nails, the hair, the eyelashes, the eyebrows and/or the scalp.

29. The photoprotective composition as defined by claim 1, formulated as a product for caring for the skin, the lips, the nails, the hair and/or the scalp.

30. The photoprotective composition as defined by claim 1, formulated as a makeup.

31. A method of formulating a combination of (i) at least one oil containing in its structure at least one amide structural unit and (ii) at least one eutectic mixture of n-butylphthalimide and of isopropylphthalimide into a cosmetic or dermatological composition containing at least one 1,3,5-triazine sunscreen, to improve the sun protection factor, the cosmetic properties and/or the stability of such composition.

32. A regime or regimen for photoprotecting the skin, the lips, the nails, the hair, the eyelashes, the eyebrows and/or the scalp against the damaging effects of UV-irradiation, comprising topically applying thereon a thus effective amount of the photoprotective composition as defined by claim 1.
